(19) United States
(12) Patent Application Publication
(10) Pub. No.: US 2026/0276568 A1
(43) Pub. Date: Sep. 17, 2026

(54) EVALUATION SYSTEM AND PREDICTIVE METHODOLOGY FOR SEDIMENTARY MICROFACIES OF SHALE OIL SWEET SPOTS IN SALINE, HYDROCARBON-RICH LACUSTRINE BASIN

(71) Applicants: Chengdu University of Technology, Chengdu (CN); State Key Laboratory of Deep Oil and Gas, China University of Petroleum (East China), Qingdao (CN)

(72) Inventors: Yiquan MA, Chengdu (CN); Fang HAO, Chengdu (CN); Yangbo LU, Chengdu (CN); Ziqi FENG, Chengdu (CN); Lei CHEN, Chengdu (CN); Yongchao LU, Chengdu (CN)

(73) Assignees: Chengdu University of Technology, Chengdu (CN); State Key Laboratory of Deep Oil and Gas, China University of Petroleum (East China), Qingdao (CN)

(21) Appl. No.: 19/404,059

(22) Filed: Dec. 1, 2025

(30) Foreign Application Priority Data
Mar. 17, 2025 (CN) 202510313331.9

(51) Int. Cl. G01N 23/20; G01N 25/22; G01N 33/24

(52) U.S. Cl. CPC G01N 23/20; G01N 25/22; G01N 33/241; G01N 2223/616

(57) ABSTRACT

The present disclosure discloses an evaluation system and a prediction method for sedimentary microfacies of salted hydrocarbon-rich lake basin shale oil sweet spots, and relates to the technical field of oil and gas exploration, comprising: a microfacies characterization module analyzing single-phase thicknesses, mineral compositions and sources, and sedimentary feature characterization parameters of salted hydrocarbon-rich lake basin fine mixed sedimentary microfacies; an oil content analysis module performing oil content analysis on the fine mixed sedimentary microfacies; an XRD mineralogical analysis module analyzing mineral compositions and contents of sedimentation samples; a multi-source data fusion analysis module comprehensively analyzing and understanding microfacies features based on a multi-source data fusion technique; and a data processing and evaluation module establishing an evaluation model based on obtained fused data, and performing predictions and grading to automatically recommend most appropriate exploration areas and mining strategies.

### TECHNICAL FIELD

The present disclosure relates to the technical field of oil and gas exploration, in particular to an evaluation system and a prediction method for sedimentary microfacies of salted hydrocarbon-rich lake basin shale oil sweet spots.

### BACKGROUND

Evaluation analysis of sedimentary microfacies of shale oil sweet spots is mainly applied in the field of oil and gas exploration, in particular for identification and evaluation of sedimentary microfacies in the oil and gas resource exploration process, and microfacies features of sediments have a crucial influence on the distribution, oil content and mining potentials of oil and gas resources. Therefore, it is of significant for establishing an efficient microfacies sweet spot evaluation system that can scientifically evaluate oil and gas potentials and predict and optimize exploration solutions based on microfacies features and associated data to improve the oil and gas exploration success rate and reduce exploration costs.

The conventional oil and gas exploration degree for salted hydrocarbon-rich lake basins is high, difficulties of increasing reserves and boosting production are great, and shale oil in the basins becomes an extremely important replacement resource. Currently, shale oil resource exploration and development for salted hydrocarbon-rich lake basins face many challenges, with obvious significances as follows:

Shale oil resources of salted hydrocarbon-rich lake basins are mainly reserved in fine mixed sediments, sedimentary microfacies of such sedimentary rock type changes rapidly, and their internal heterogeneity is very strong, resulting in poor recognition and difficult predictions of sedimentary microfacies favorable for shale oil enrichment, and causing significant problems such as unknown shale oil enrichment mechanisms. For example, in fine mixed sediments of Jiyang Depression Paleogene System Es₃^{x}-Es₄^{s}, the carbonate minerals have a large range of contents and distribution (11%-95%), are highly mixed with chip components of clay minerals, quartz and feldspar in different proportions, alternately superimposed and mixed in the form of grain layer/layers, or mixed in the form of uniform mixing, and it is very spatially variable, are extremely large in spatial changes, and pose great challenges for identification, tracking and predictions of the sedimentary microfacies of shale oil sweet spots.

Moreover, due to the complex geological conditions, large burial depth, uneven resource distribution, and high technical requirements for exploration and development, the exploration and development of shale oil of salted hydrocarbon-rich lake basins are extremely challenging and costly. For example, the use of techniques such as risky exploratory drilling, horizontal drilling, large-scale fracturing revamping, and underground in-situ heating conversion requires significant investment of personnel, techniques and capital, increasing exploration and development costs.

Currently, traditional oil and gas exploration methods rely on a single data source, the diversity of sediments and the variation in spatial distribution of microfacies features tend to be ignored, this results in limitations of the evaluation model and degradation of prediction accuracy, and thus, it is difficult to meet the need to improve prediction accuracy of sweet spots in the shale oil exploration and development process of the salted hydrocarbon-rich lake basins. To solve this problem, the present disclosure provides a microfacies evaluation system and prediction method for shale oil exploration and development features of the salted hydrocarbon-rich lake basins; and in combination with multiple advanced techniques such as multi-source data fusion, high-precision microfacies characterization, oil content analysis, and mineralogical analysis, the microfacies features of the sediments are comprehensively and accurately analyzed, oil and gas potentials are evaluated, and exploration decision support is provided.

### SUMMARY

To solve the above technical problems, an evaluation system and a prediction method for sedimentary microfacies of salted hydrocarbon-rich lake basin shale oil sweet spots are provided, and the present technical solution solves the above problems.

In order to achieve the above object, the present disclosure adopts the following technical solutions:

An evaluation system and a prediction method for sedimentary microfacies of salted hydrocarbon-rich lake basin shale oil sweet spots, including:
a microfacies characterization module: the microfacies characterization module is used for analyzing single-phase thicknesses, mineralogical features and mineral sources, and sedimentary feature characterization parameters of fine mixed sedimentary microfacies, and identifying sedimentation types, bedding features, and sedimentation environments;
an oil content analysis module: the oil content analysis module is used for performing oil content analysis on the fine mixed sedimentary microfacies, including parameter analysis of total organic carbon and oil saturation, and evaluating a potential of oil and gas resources;
an XRD (X-ray diffraction) mineralogical analysis module: the XRD mineralogical analysis module is used for analyzing mineral compositions of sedimentation samples, and identifying contents and distribution of felsic minerals and clay minerals as well as carbonate minerals;
a multi-source data fusion analysis module: the multi-source data fusion analysis module is electrically connected to the XRD mineralogical analysis module, the oil content analysis module, and the microfacies characterization module, and the multi-source data fusion analysis module is used for comprehensively analyzing and understanding microfacies features, including remote sensing data, seismic data, drilling data, and geological survey data, based on a multi-source data fusion technique; and
a data processing and evaluation module: the data processing and evaluation module is electrically connected to the multi-source data fusion analysis module, and the data processing and evaluation module is used for establishing evaluation models of favorable zones, sweet spot sections and sweet spot areas of the fine mixed microfacies according to obtained fusion data of microfacies characterization parameters, oil content analysis, and mineralogical data, and performing predictions and classifications to automatically recommend most suitable exploration areas, drilling depths, and mining strategies.

Preferably, the microfacies characterization module specifically includes:
using an Itrax series ultrahigh-resolution core element scanner cooperatively researched and developed by Sweden and UK to perform high-precision physical and chemical feature analysis on the sedimentation samples to obtain ultrahigh-resolution (2 mm apart) element content and ratio data and obtain fine quantitative results characterizing various key elements of rock brittleness and sedimentation environments, such as changes reflecting rock brittle elements, climatic background conditions, does the volcano have any impact, redox conditions of water bodies, paleoproductivity, water body salinity, land source inputs, does hydrothermal solutions have any impact, etc.;
using a thin slice microscope to observe and study mineralogical features of the sedimentation samples, including morphology, size distribution and sedimentation features of mineral particles, to obtain mineral composition and distribution features and their sources, including exogenous minerals, endogenous minerals or mixed sources, and analyze their associations with the sedimentation environments;
using a thin slice microscope to observe bedding, configurations and microstructures of a settled layer, performing sedimentation feature analysis in combination with stratigraphic sections, identifying sedimentation structure types, including grain layer structures, laminar structures, block structures, deformation structures, lenticular structures, and microcut structures, and presuming the sedimentation environments of sediments;
according to comprehensive features, confirming specific sedimentation types of the sediments and performing microfacies partitioning to summarize ultrahigh-resolution element response features of different sedimentary microfacies, according to the identified sedimentation types and microfacies, inferring ancient sedimentation environments in combination with modern sedimentation environment features, adopting environment indicating minerals as signs of environment changes, and further confirming the sedimentation environments; and
based on the microfacies feature data, performing microfacies spatial distribution analysis, based on a stratigraphic histogram and a deep line graph tool, studying spatial evolution of microfacies under different sedimentation environments to obtain a dynamic analysis diagram of microfacies distribution and evolution of the sedimentation environments, and identifying change trends of the sedimentation environments.

Preferably, the oil content analysis module specifically includes:
based on a high-temperature combustion method, performing total organic carbon analysis on samples to obtain a TOC (Total Organic Carbon) value of each sample, and analyzing soluble organic matters in the samples through a solvent extraction method;
based on oil and gas yields and hydrocarbon generation potentials, obtaining potential oil and gas generation zones, and estimating a potential of minable oil and gas resources;
performing hydrogen-carbon ratio analysis on the organic matters in the samples to judge their hydrocarbon generation potentials, and further evaluating whether the sediments are suitable for generating oil and gas through an H/C ratio; and
analyzing types, maturity and oil and gas generation capabilities of the organic matters in the samples through pyrolysis curves, and clarifying generation environments and types of oil and gas resources through organic matter type analysis.

Preferably, the XRD mineralogical analysis module specifically includes:
placing prepared samples into an XRD instrument for diffraction scanning, allowing X-rays to pass through the samples and interact with crystal lattices in minerals to generate a diffraction pattern, and identifying types of minerals present in the samples by matching diffraction peak positions in the pattern with diffraction data of known minerals; and
performing quantitative mineral analysis through position, intensity, width, and integrated area information of diffraction peaks to quantitatively identify species, distribution, and relative contents of felsic minerals, carbonate minerals, and clay minerals in the samples.

Preferably, the multi-source data fusion analysis module specifically includes:
pre-processing the remote sensing data, including geometry correction, radiometric correction, de-noising, and data resampling, and performing orthorectification on remote-sensing images;
pre-processing the seismic data, including de-noising, velocity model construction, and seismic inversion, etc., and performing de-noising and signal enhancement processing on the seismic data;
pre-processing the drilling data, including data screening, outlier removal, and missing value imputation, and pre-processing the geological survey data, including data standardization, raw geological record processing, and geological feature extraction;
based on a principal component analysis (PCA) algorithm, extracting remote sensing data features, extracting reflecting layer, strata thickness, elastic modulus, velocity and density features from the seismic wave data, and performing feature extraction on the drilling data and the geological survey data;
where a formula of the PCA algorithm is:

$$ζ = \frac{1}{n - 1} {\sum }_{i = 1}^{n} ( {x}_{i} - \overline{x} ) {( {x}_{i} - \overline{x} )}^{T}$$

in the formula, xᵢ is ith sample data, x̅ is a sample mean, n is a number of samples, ζ is a covariance matrix, and T is a transpose;
based on a Kalman filtering algorithm, fusing data from different sources;
identifying microfacies features of the sediments through fused data in combination with geological models, analyzing microfacies feature distribution in different regions and different depths to reveal sedimentation environments, sedimentation patterns, and oil and gas potentials, and evaluating distribution, reserves and mining potentials of oil and gas reservoirs based on microfacies analysis, where a formula of microfacies classifying is:

$$min {\sum }_{i = 1}^{n} {\sum }_{j = 1}^{k} rij {‖ {x}_{i} - {c}_{j} ‖}^{2}$$

in the formula, rij is a membership between sample i and type j, xⱼ is sample data, cⱼ is a center of types, k is a number of types, and n is a number of samples; and
presenting analysis results in a form of graphs or three-dimensional visualizations to generate microfacies distribution maps and heat maps, and giving a comprehensive interpretation of microfacies features based on analysis results of the fused data to reveal their relationships with oil and gas reservoir distribution and reservoir properties.

Preferably, based on a Kalman filtering algorithm, fusing data from different sources includes:
where the formula of the Kalman filtering algorithm is:

$$\hat{x} k \vert k = \hat{x} k \vert k - 1 + {K}_{k} ( {y}_{k} - {H}_{k} \hat{x} k \vert k - 1 )$$

in the formula, îk|k−1 is a predicted value at a previous moment, Kₖ is a Kalman gain, yₖ is an observed value, and Hₖ is an observation matrix.

Preferably, the data processing and evaluation module specifically includes: based on the fused data, establishing an evaluation model of different regions
using a statistical analysis method model;
ranking the regions based on a support vector machine according to results of the evaluation model;
making predictions of future regions based on an established evaluation model; and
automatically recommending a most suitable exploration area according to a score of the evaluation model, recommending an optimal drilling depth based on microfacies distribution and oil and gas content predictions, and recommending a best mining strategy according to oil and gas distribution and reserve factors.

Preferably, based on the fused data, establishing an evaluation model of different regions using a statistical analysis method model specifically includes:
where a formula of the evaluation model is:

$$S = {\sum }_{i = 1}^{m} {w}_{i} \cdot {f}_{i} ( x )$$

in the formula, S is a comprehensive score of a region, i is an index variable, m is a number of features, wᵢ is a weight coefficient, fᵢ(x) is a value of an i feature, and x is feature data.

An evaluation system and a prediction method for sedimentary microfacies of salted hydrocarbon-rich lake basin shale oil sweet spots, including:
acquiring relevant characterization parameters, oil content analysis data, and XRD mineralogical analysis data of fine mixed sedimentary microfacies;
determining microfacies types, bedding features, and their sedimentation environments according to the data;
performing oil content predictions on different microfacies, evaluating oil and gas potentials in combination with sedimentation features, and positioning favorable zones, sweet spot sections, and sweet spot areas;
using a regression analysis method to establish a quantitative relationship model between oil and gas contents and microfacies types to perform further oil and gas distribution predictions; and
verifying accuracy of prediction results through comparison with actual results of exploration wells based on existing exploration well data.

Preferably, using a regression analysis method to establish a quantitative relationship model between oil and gas contents and microfacies types to perform further oil and gas distribution predictions specifically includes:
where a prediction formula is:

$$\hat{y} = {γ}_{0} + {γ}_{1} {x}_{1} + {γ}_{2} {x}_{2} + … + {γ}_{n} {x}_{n}$$

in the formula, ŷ is a predicted value, x₁x₂ . . . xₙ is an input feature, and γ₀γ₁γ₂ . . . γₙ is a regression coefficient.

Compared with the prior art, the beneficial effects of the present disclosure are:

The present disclosure proposes to combine a variety of technological means such as thickness analysis, mineral composition and source analysis, and oil content analysis to improve the oil and gas resource evaluation accuracy; systematically and effectively integrates different data sources through multi-source data fusion analysis to optimize selection and drilling depths of exploration zones; predicts oil and gas resource potentials based on regression analysis to further improve prediction accuracy; combines a support vector machine algorithm to automatically recommend optimal exploration areas, drilling depths and exploitation strategies; and is strong in system adaptability, can handle multiple data sources, is applicable to different regions and exploration environments, and improves efficiency and success rate of oil and gas exploration.

### BRIEF DESCRIPTION OF THE DRAWINGS

FIG. 1 is a system framework diagram of the present disclosure; and

FIG. 2 is a flow frame diagram of a method according to the present disclosure.

### DETAILED DESCRIPTION OF THE EMBODIMENTS

The following description is used for disclosing the present disclosure to enable those skilled in the art to practice the present disclosure. The preferred embodiments in the following description are given by way of examples only, and other obvious variations will occur to those skilled in the art.

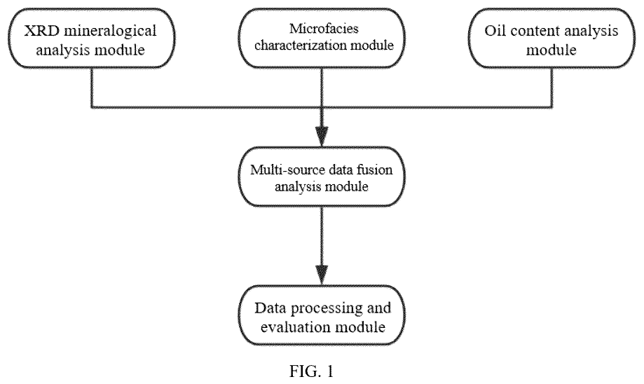

Referring to FIG. 1, an evaluation system and a prediction method for sedimentary microfacies of salted hydrocarbon-rich lake basin shale oil sweet spots, including:

### Microfacies Characterization Module:

using an Itrax series ultrahigh-resolution core element scanner cooperatively researched and developed by Sweden and UK to perform high-precision physical and chemical feature analysis on the sedimentation samples under test conditions of 30 kv, 30 mA, 30 s, and a scanning area of 6 mm (width)×2 mm (length) with the sensitivity up to a ppm-level (one part per million) detection range to obtain ultrahigh-resolution (2 mm apart) element content and ratio data and obtain fine quantitative results characterizing various key elements of rock brittleness and sedimentation environments, such as changes reflecting rock brittle elements, climatic background conditions, does the volcano have any impact, redox conditions of water bodies, paleoproductivity, water body salinity, land source inputs, does hydrothermal solutions have any impact, etc.;
using a thin slice microscope to observe and study mineralogical features of the sedimentation samples, including morphology, size distribution and sedimentation features of mineral particles, to obtain mineral composition and distribution features and their sources (exogenous minerals, endogenous minerals or mixed sources), and analyze their associations with the sedimentation environments;
using a thin slice microscope to observe bedding, sedimentation configurations and microstructures of a settled layer, performing sedimentation feature analysis in combination with stratigraphic sections, identifying sedimentation structure types, including grain layer structures, cross bedding layered structures, wave-shaped bedding block structures, deformation structures, lenticular structures, and microcut structures, and presuming the sedimentation environments of sediments;
according to comprehensive features, confirming specific sedimentation types of the sediments and performing microfacies partitioning to summarize ultrahigh-resolution element response features of different sedimentary microfacies, according to the identified sedimentation types and microfacies, inferring ancient sedimentation environments in combination with modern sedimentation environment features, adopting environment indicating minerals as signs of environment changes, and further confirming the sedimentation environments; and
based on the microfacies feature data, performing microfacies spatial distribution analysis, based on a stratigraphic histogram and a deep line graph tool, studying spatial evolution of microfacies under different sedimentation environments to obtain a dynamic analysis diagram of microfacies distribution and evolution of the sedimentation environments, and identifying change trends of the sedimentation environments.

### Oil Content Analysis Module:

based on a high-temperature combustion method, performing total organic carbon analysis on samples to obtain a TOC value of each sample, and analyzing soluble organic matters in the samples through a solvent extraction method;
based on oil and gas yields and hydrocarbon generation potentials, obtaining potential oil and gas generation zones, and estimating a potential of minable oil and gas resources;
performing hydrogen-carbon ratio analysis on the organic matters in the samples to judge their hydrocarbon generation potentials, and further evaluating whether the sediments are suitable for generating oil and gas through an H/C ratio; and
analyzing types, maturity and oil and gas generation capabilities of the organic matters in the samples through pyrolysis curves, and clarifying generation environments and types of oil and gas resources through organic matter type analysis.

Analyzing types, maturity and oil and gas generation capabilities of the organic matters in the samples through pyrolysis curves can be carried out by the following steps:

### 1. Organic Matter Type Analysis

Parameters: hydrogen index (HI) and maximum pyrolysis peak temperature (Tmax)

Method: a panel of HI and Tmax is used for classifying organic matter types. In general, HI represents an oil generation potential of organic matters, and Tmax represents thermal maturity of the organic matters.

Panel: a classical panel classifies the organic matters into different types, such as a humus-silt type (III) and a humus-silt type (II2), etc. By plotting HI and Tmax data points on the panel, the types of the organic matters can be determined.

### 2. Maturity Analysis

Parameter: maximum pyrolysis peak temperature (Tmax)

Method: Tmax is the temperature at which kerogen cracks to produce the maximum hydrocarbon content and can be used for indicating the thermal maturity of the organic matters.

### Maturity Range:

−400° C.-430° C.: immature organic matters
−430° C.-450° C.: mature organic matters (oil generation window)
−450° C.-500° C.: highly mature organic matters (gas generation window)

Vitrinite reflectance (Ro): maturity can also be further confirmed by the relationship between Tmax and Ro. For example, Ro=0.5%-1.0% represents mature organic matters, and Ro>1.0% represents highly mature organic matters.

### 3. Oil and Gas Generation Capacity Analysis

Parameters: S1 (amount of free hydrocarbons), S2 (amount of pyrolyzed hydrocarbons), and S3 (amount of CO₂ produced by pyrolysis of organic matters)

### Method:

S1: represents the content of free hydrocarbons in a sample, typically reflecting the content of light hydrocarbons.
S2: represents the content of hydrocarbons released by kerogen during cracking and is an important parameter for evaluating an oil generation potential.
S3: represents the amount of CO₂ produced by pyrolysis of organic matters, which can reflect the degree of oxidation of organic matters.

Oil generation potential (Pg): the oil generation potential is calculated by S1 and S2, Pg=S1+S2, which can evaluate the oil generation potential of strata.

### 4. Organic Matter Type and Oil and Gas Resource Generation Environments and Types

Humus-silt type (II1): generally represents a lacustrine facies sedimentation environment, which has a high oil generation potential and is mainly used for crude oil generation.

Humus-silt type (II 2): generally represents a marine facies sedimentation environment, which has a medium oil generation potential and is mainly used for light oil and natural gas generation.

Type index (IL): the origin and type of organic matters can be further confirmed by IL. IL=S2/(S1+S2), the higher the IL value, the greater oil generation potential of the organic matters.

### Integrated Analysis

With the above parameters and methods, the types of organic matters, maturity, and oil and gas generating capacities of the shale samples can be analyzed comprehensively. The specific steps are as follows:
1. Determination of parameters: a rock pyrolyzer is used for determining parameters such as S1, S2, S3, Tmax, and HI.
2. Plotting of panel: HI and Tmax data points are plotted on the panel to determine the types of the organic matters.
3. Evaluation of maturity: the maturity of the organic matters is evaluated based on the Tmax and Ro values.
4. Calculation of oil generation potential: the oil generation potential is calculated by S1 and S2 to evaluate the oil generation potential.
5. Determination of generation environment: the generation environment and type of oil and gas resources are determined based on the organic matter type and type index.

### XRD Mineralogical Analysis Module:

placing prepared samples into an XRD instrument for diffraction scanning, allowing X-rays to pass through the samples and interact with crystal lattices in minerals to generate a diffraction pattern, and identifying types of minerals present in the samples by matching diffraction peak positions in the pattern with diffraction data of known minerals; and
performing quantitative mineral analysis through position, intensity, width, and integrated area information of diffraction peaks to quantitatively identify species, distribution, and relative contents of felsic minerals, carbonate minerals, and clay minerals in the samples.

### Multi-Source Data Fusion Analysis Module:

pre-processing the remote sensing data, including geometry correction, radiometric correction, de-noising, and data resampling, and performing orthorectification on remote-sensing images;
pre-processing the seismic data, including de-noising, velocity model construction, and seismic inversion, etc., and performing de-noising and signal enhancement processing on the seismic data;
pre-processing the drilling data, including data screening, outlier removal, and missing value imputation, and pre-processing the geological survey data, including data standardization, raw geological record processing, and geological feature extraction;
based on a PCA algorithm, extracting remote sensing data features, extracting reflecting layer, strata thickness, elastic modulus, velocity and density features from the seismic wave data, and performing feature extraction on the drilling data and the geological survey data;
where a formula of the PCA algorithm is:

$$ζ = \frac{1}{n - 1} {\sum }_{i = 1}^{n} ( {x}_{i} - \overline{x} ) {( {x}_{i} - \overline{x} )}^{T}$$

in the formula, xᵢ is ith sample data, x̅ is a sample mean, n is a number of samples, ζ is a covariance matrix, and T is a transpose;
based on a Kalman filtering algorithm, fusing data from different sources;
where the formula of the Kalman filtering algorithm is:

$$\hat{x} k \vert k = \hat{x} k \vert k - 1 + {K}_{k} ( {y}_{k} - {H}_{k} \hat{x} k \vert k - 1 )$$

in the formula, \hat{x}k|k−1 is a predicted value at a previous moment, Kₖ is a Kalman gain, yₖ is an observed value, and Hₖ is an observation matrix;
identifying microfacies features of the sediments through fused data in combination with geological models, analyzing microfacies feature distribution in different regions and different depths to reveal sedimentation environments, sedimentation patterns, and oil and gas potentials, and evaluating distribution, reserves and mining potentials of oil and gas reservoirs based on microfacies analysis, where a formula of microfacies classifying is:

$$min {\sum }_{i = 1}^{n} {\sum }_{j = 1}^{k} rij {‖ {x}_{i} - {c}_{j} ‖}^{2}$$

in the formula, rij is a membership between sample i and type j, xᵢ is sample data, cⱼ is a center of types, k is a number of types, and n is a number of samples; and
presenting analysis results in a form of graphs or three-dimensional visualizations to generate microfacies distribution maps and heat maps, and giving a comprehensive interpretation of microfacies features based on analysis results of the fused data to reveal their relationships with oil and gas reservoir distribution and reservoir properties.

### Data Processing and Evaluation Module:

based on the fused data, establishing an evaluation model of different regions using a statistical analysis method model;
where a formula of the evaluation model is:

$$S = {\sum }_{i = 1}^{m} {w}_{i} \cdot {f}_{i} ( x )$$

in the formula, S is a comprehensive score of a region, i is an index variable, m is a number of features, wᵢ is a weight coefficient, fᵢ(x) is a value of an i feature, and x is feature data;
ranking the regions based on a support vector machine according to results of the evaluation model;
making predictions of future regions based on an established evaluation model; and
automatically recommending a most suitable exploration area according to a score of the evaluation model, recommending an optimal drilling depth based on microfacies distribution and oil and gas content predictions, and recommending a best mining strategy according to oil and gas distribution and reserve factors.

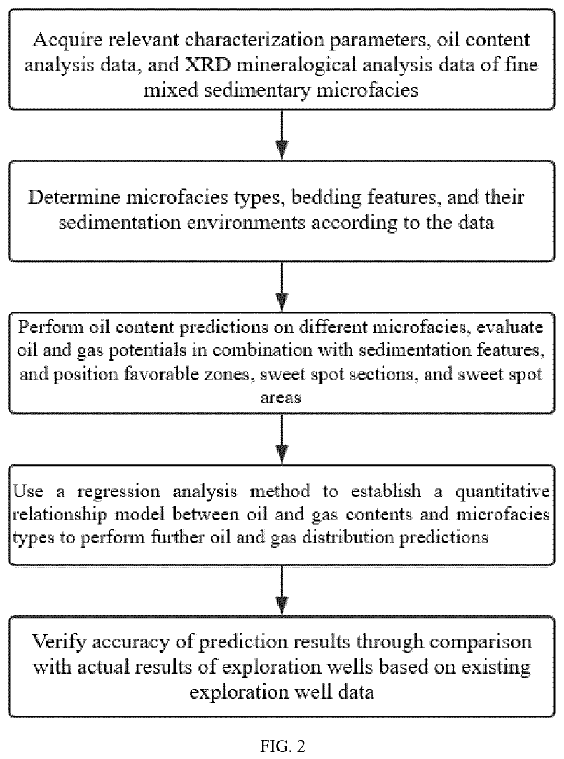

Referring to FIG. 2, an evaluation system and a prediction method for sedimentary microfacies of salted hydrocarbon-rich lake basin shale oil sweet spots, including:
Step 1: acquiring relevant characterization parameters, oil content analysis data, and XRD mineralogical analysis data of fine mixed sedimentary microfacies;
Step 2: determining microfacies types, bedding features and their sedimentation environments according to the data;
Step 3: performing oil content predictions on different microfacies, evaluating oil and gas potentials in combination with sedimentation features, and positioning favorable zones, sweet spot sections, and sweet spot areas;
Step 4: using a regression analysis method to establish a quantitative relationship model between oil and gas contents and microfacies types to perform further oil and gas distribution predictions;
where a prediction formula is:

$$\hat{y} = {γ}_{0} + {γ}_{1} {x}_{1} + {γ}_{2} {x}_{2} + … + {γ}_{n} {x}_{n}$$

in the formula, ŷ is a predicted value, x₁x₂ . . . xₙ is an input feature, and γ₀γ₁γ₂ . . . γₙ is a regression coefficient.
Step 5: verifying accuracy of prediction results through comparison with actual results of exploration wells based on existing exploration well data.

In summary, the present disclosure has the following advantages:
By combining various technical means such as thickness analysis, mineral composition and source analysis, and oil content analysis, the system can comprehensively characterize microfacies features of sediments and effectively improve evaluation precision of oil and gas resources; meanwhile, the system integrates a multi-source data fusion analysis module, thereby being capable of spanning barriers of different data sources to achieve comprehensive utilization of data and improve the comprehensive analysis capability of the system;
by analyzing the spatial distribution of microfacies features, the system is capable of revealing evolution trends of microfacies under different sedimentation environments, providing visualization and dynamic analysis of the association of microfacies with the distribution of oil and gas resources, and further helping to optimize the selection of exploration zones and the determination of the drilling depth;
the system establishes a quantitative relationship model between oil and gas contents and microfacies types through a regression analysis method, and is capable of effectively predicting oil and gas resource potentials of different microfacies regions, thereby providing scientific basis for oil and gas distribution and reserve predictions; through verification on exploration well data, the system is capable of further improving accuracy of predictions;
the data processing and evaluation module can automatically generate suitable exploration areas, drilling depths and mining strategies according to microfacies analysis results; in combination with the support vector machine algorithm, the system can automatically recommend optimal exploration areas according to the scores of the evaluation model, ensuring the efficiency of exploration activities and maximum utilization of resources; and
the system is capable of processing information from different data sources, has strong adaptability, and is suitable for the oil and gas exploration needs in different regions and in different exploration environments.

The foregoing has shown and described the basic principles, main features and advantages of the present disclosure. It will be appreciated by those skilled in the art that the present disclosure is not limited to the above-described embodiments, those described in the embodiments and the specification are merely the principles of the present disclosure, and various changes and modifications of the present disclosure are possible without departing from the spirit and scope of the present disclosure, which fall within the scope of the present disclosure as claimed. The scope of protection claimed herein is defined by the claims attached and their equivalents.

What is claimed is:

1. An evaluation system for sedimentary microfacies of salted hydrocarbon-rich lake basin shale oil sweet spots, comprising:
a microfacies characterization module: the microfacies characterization module is used for analyzing single-phase thicknesses, mineralogical features and mineral sources, sedimentary feature characterization parameters, and high-precision physical and chemical features of fine mixed sedimentary microfacies, and identifying refined sedimentation types, bedding features, and sedimentation environments;
an oil content analysis module: the oil content analysis module is used for performing oil content analysis on the fine mixed sedimentary microfacies, including parameter analysis of total organic carbon and oil saturation, and evaluating a potential of oil and gas resources;
an XRD (X-ray diffraction) mineralogical analysis module: the XRD mineralogical analysis module is used for analyzing mineral compositions of sedimentation samples, and identifying contents and distribution of felsic minerals and clay minerals as well as carbonate minerals;
a multi-source data fusion analysis module: the multi-source data fusion analysis module is electrically connected to the XRD mineralogical analysis module, the oil content analysis module, and the microfacies characterization module, and the multi-source data fusion analysis module is used for comprehensively analyzing and understanding microfacies features, including remote sensing data, seismic data, drilling data, and geological survey data, based on a multi-source data fusion technique; and
a data processing and evaluation module: the data processing and evaluation module is electrically connected to the multi-source data fusion analysis module, and the data processing and evaluation module is used for establishing evaluation models of favorable zones, sweet spot sections and sweet spot areas of the fine mixed microfacies according to obtained fusion data of microfacies characterization parameters, oil content analysis, and mineralogical data, and performing predictions and classifications to recommend most suitable exploration areas, drilling depths, and mining strategies.

2. The evaluation system for sedimentary microfacies of salted hydrocarbon-rich lake basin shale oil sweet spots according to claim 1, wherein the microfacies characterization module comprises:
using an Itrax series ultrahigh-resolution core element scanner cooperatively researched and developed by Sweden and UK to perform high-precision physical and chemical feature analysis on the sedimentation samples to obtain ultrahigh-resolution element content and ratio data and obtain fine quantitative results characterizing various key elements of rock brittleness and sedimentation environments;
using a thin slice under a microscope to observe and study mineralogical features of the sedimentation samples, including morphology, size distribution and sedimentation features of mineral particles, to obtain mineral composition and distribution features and their sources and analyze their associations with the sedimentation environments;
using a thin slice under a microscope to further observe bedding, sedimentation configurations and microstructures of a settled layer, performing sedimentation feature analysis in combination with stratigraphic sections, identifying sedimentation structure types, including grain layer structures, laminar structures, block structures, deformation structures, lenticular structures, and microcut structures, and presuming the sedimentation environments of sediments;
according to comprehensive features, confirming specific sedimentation types of the sediments and performing microfacies partitioning to summarize ultrahigh-resolution element response features of different sedimentary microfacies, according to the identified sedimentation types and microfacies, inferring ancient sedimentation environments in combination with modern sedimentation environment features, adopting environment indicating minerals as signs of environment changes, and further confirming the sedimentation environments; and
based on the microfacies feature data, performing microfacies spatial distribution analysis, based on a stratigraphic histogram and a deep line graph tool, studying spatial evolution of microfacies under different sedimentation environments to obtain a dynamic analysis diagram of microfacies distribution and evolution of the sedimentation environments, and identifying change trends of the sedimentation environments.

3. The evaluation system for sedimentary microfacies of salted hydrocarbon-rich lake basin shale oil sweet spots according to claim 2, wherein the oil content analysis module comprises:
based on a high-temperature combustion method, performing total organic carbon analysis on samples to obtain a TOC (Total Organic Carbon) value of each sample, and analyzing soluble organic matters in the samples through a solvent extraction method;
based on oil and gas yields and hydrocarbon generation potentials, obtaining potential oil and gas generation zones, and estimating a potential of minable oil and gas resources;
performing hydrogen-carbon ratio analysis on the organic matters in the samples to judge their hydrocarbon generation potentials, and further evaluating whether the sediments are suitable for generating oil and gas through an H/C ratio (ratio of a number of hydrogen atoms to a number of carbon atoms in the matters); and
analyzing types, maturity and oil and gas generation capabilities of the organic matters in the samples through pyrolysis curves, and clarifying generation environments and types of oil and gas resources through organic matter type analysis.

4. The evaluation system for sedimentary microfacies of salted hydrocarbon-rich lake basin shale oil sweet spots according to claim 3, wherein the XRD mineralogical analysis module comprises:
placing prepared samples into an XRD instrument for diffraction scanning, allowing X-rays to pass through the samples and interact with crystal lattices in minerals to generate a diffraction pattern, and identifying types of minerals present in the samples by matching diffraction peak positions in the pattern with diffraction data of known minerals; and
performing quantitative mineral analysis through position, intensity, width, and integrated area information of diffraction peaks to quantitatively identify species, distribution, and contents of felsic minerals, carbonate minerals, and clay minerals in the samples.

5. The evaluation system for sedimentary microfacies of salted hydrocarbon-rich lake basin shale oil sweet spots according to claim 4, wherein the multi-source data fusion analysis module comprises:
pre-processing the remote sensing data, including geometry correction, radiometric correction, de-noising, and data resampling, and performing orthorectification on remote-sensing images;
pre-processing the seismic data, including de-noising, velocity model construction, and seismic inversion, etc., and performing de-noising and signal enhancement processing on the seismic data;
pre-processing the drilling data, including data screening, outlier removal, and missing value imputation, and pre-processing the geological survey data, including data standardization, raw geological record processing, and geological feature extraction;
based on a principal component analysis (PCA) algorithm, extracting remote sensing data features, extracting reflecting layer, strata thickness, elastic modulus, velocity and density features from the seismic wave data, and performing feature extraction on the drilling data and the geological survey data;
wherein a formula of the PCA algorithm is:
$$ζ = \frac{1}{n - 1} {\sum }_{i = 1}^{n} ( {x}_{i} - \overline{x} ) {( {x}_{i} - \overline{x} )}^{T}$$
in the formula, xᵢ is ith sample data, x̅ is a sample mean, n is a number of samples, ζ is a covariance matrix, and T is a transpose;
based on a Kalman filtering algorithm, fusing data from different sources;
identifying microfacies features of the sediments through fused data in combination with geological models, analyzing microfacies feature distribution in different regions and different depths to reveal sedimentation environments, sedimentation patterns, and oil and gas potentials, and evaluating distribution, reserves and mining potentials of oil and gas reservoirs based on microfacies analysis, wherein a formula of microfacies classifying is:
$$min {\sum }_{i = 1}^{n} {\sum }_{j = 1}^{k} rij {‖ {x}_{i} - {c}_{j} ‖}^{2}$$
in the formula, rij is a membership between sample i and type j, xᵢ is sample data, cⱼ is a center of types, k is a number of types, and n is a number of samples; and
presenting analysis results in a form of graphs or three-dimensional visualizations to generate microfacies distribution maps and heat maps.

6. The evaluation system for sedimentary microfacies of salted hydrocarbon-rich lake basin shale oil sweet spots according to claim 5, wherein based on a Kalman filtering algorithm, fusing data from different sources comprises:
wherein the formula of the Kalman filtering algorithm is:
$$\hat{x} k \vert k = \hat{x} k \vert k - 1 + {K}_{k} ( {y}_{k} - {H}_{k} \hat{x} k \vert k - 1 )$$
in the formula, \hat{x}k|k−1 is a predicted value at a previous moment, Kₖ is a Kalman gain, yₖ is an observed value, and Hₖ is an observation matrix.

7. The evaluation system for sedimentary microfacies of salted hydrocarbon-rich lake basin shale oil sweet spots according to claim 6, wherein the data processing and evaluation module comprises:
based on the fused data, establishing an evaluation model of different regions using a statistical analysis method model;
ranking the regions based on a support vector machine according to results of the evaluation model;
making predictions of future regions based on an established evaluation model; and
automatically recommending a most suitable exploration area according to a score of the evaluation model, recommending an optimal drilling depth based on microfacies distribution and oil and gas content predictions, and recommending a best mining strategy according to oil and gas distribution and reserve factors.

8. The evaluation system for sedimentary microfacies of salted hydrocarbon-rich lake basin shale oil sweet spots according to claim 7, wherein based on the fused data, establishing an evaluation model of different regions using a statistical analysis method model comprises:
wherein a formula of the evaluation model is:
$$S = {\sum }_{i = 1}^{m} {w}_{i} \cdot {f}_{i} ( x )$$
in the formula, S is a comprehensive score of a region, i is an index variable, m is a number of features, wᵢ is a weight coefficient, fᵢ(x) is a value of an i feature, and x is feature data.

9. A prediction method for sedimentary microfacies of salted hydrocarbon-rich lake basin shale oil sweet spots, comprising:
acquiring relevant characterization parameters, oil content analysis data, and XRD mineralogical analysis data of fine mixed sedimentary microfacies;
determining microfacies types, bedding features, and their sedimentation environments according to the data;
performing oil content predictions on different microfacies, evaluating oil and gas potentials in combination with sedimentation features, and positioning favorable zones, sweet spot sections, and sweet spot areas;
using a regression analysis method to establish a quantitative relationship model between oil and gas contents and microfacies types to perform further oil and gas distribution predictions; and
verifying accuracy of prediction results through comparison with actual results of exploration wells based on existing exploration well data.

10. The prediction method for sedimentary microfacies of salted hydrocarbon-rich lake basin shale oil sweet spots according to claim 9, wherein using a regression analysis method to establish a quantitative relationship model between oil and gas contents and microfacies types to perform further oil and gas distribution predictions comprises:
wherein a prediction formula is:
$$\hat{y} = {γ}_{0} + {γ}_{1} {x}_{1} + {γ}_{2} {x}_{2} + … + {γ}_{n} {x}_{n}$$
in the formula, ŷ is a predicted value, x₁x₂ . . . xₙ is an input feature, and γ₀γ₁γ₂ . . . γₙ is a regression coefficient.
